(19) United States
(12) Patent Application Publication
(10) Pub. No.: US 2004/0071649 A1
(43) Pub. Date: Apr. 15, 2004

(54) Use of alkylpolyxylosides as emulsifying agents for preparing water-in-oil emulsions

(76) Inventors: Chantal Amalric, Blan (FR); Alicia Roso, Saix (FR); Nelly Michel, Maisons Alfort (FR); Guy Tabacchi, Castres (FR); Alain Milius, Nice (FR)

Correspondence Address: DENNISON, SCHULTZ, DOUGHERTY & MACDONALD, 1727 KING STREET, SUITE 105, ALEXANDRIA, VA, 22314, US

(21) Appl. No.: 10/466,558

(22) PCT Filed: Feb. 5, 2002
(86) PCT No.: PCT/FR02/00431

(30) Foreign Application Priority Data
Feb. 5, 2001 (FR) 01/01480
Oct. 5, 2001 (FR) 01/12878

(51) Int. Cl. A61K 7/06; A61K 7/11

(52) U.S. Cl. 424/70.13

(57) ABSTRACT

The invention relates to the use of an alkylpolyxyloside obtained from a Guerbet alcohol having from 16 to 28 carbon atoms, as an emulsifier for the preparation of water-in-oil emulsions.

The invention further relates to the water-in-oil emulsions containing such an alkylpolyxyloside.

The present invention relates to the use of particular alkylpolyxylosides as emulsifiers for the preparation of water-in-oil emulsions, and to the water-in-oil emulsions containing them.

The invention is applicable especially to the field of cosmetics.

Patent application FR 00 04414, filed on 6 Apr. 2000, describes compounds of the formula

R—O—(X)ₚ

in which:

p is a decimal number between 1 and 5,

X is the xylose residue, and

R is a branched alkyl radical of the formula

CH(CₙH₂ₙ₊₁)(CₘH₂ₘ₊₁)—CH₂—

in which m is an integer between 6 and 18, n is an integer between 4 and 18 and the sum n+m is greater than or equal to 10.

These compounds are useful as surfactants.

Furthermore, examples of alkylpolyglucosides obtained from a fatty alcohol are described in patent application FR-A-2 790 977.

It is apparent from these examples that only alkylpolyglucosides obtained from oleyl and/or isostearyl alcohol make it possible to obtain an emulsion of the water-in-oil type. Other alkylpolyglucosides, especially those obtained from isooctadecyl alcohol and a Guerbet alcohol possessing 18 carbon atoms, yield emulsions of the oil-in-water type.

It has now been discovered, contrary to all expectations, that alkylpolyxylosides obtained from a Guerbet alcohol having from 16 to 28 carbon atoms make it possible to obtain stable water-in-oil emulsions.

Thus, according to a first feature, the invention relates to the use of an alkylpolyxyloside of the formula

R—O—(X)ₚ (I)

in which:

p is a decimal number between 1 and 5,

X is the xylose residue, and

R is a branched alkyl radical of the formula

CH(CₙH₂ₙ₊₁)(CₘH₂ₘ₊₁)—CH₂—

in which m is an integer between 6 and 12, n is an integer between 8 and 14 and the sum n+m ranges from 14 to 26,

or of a composition consisting of a mixture of at least two compounds as defined above,

as an emulsifier for the preparation of water-in-oil emulsions.

Preferably, the sum n+m is equal to 14, 16, 18, 22 or 26 and R is more particularly a 2-hexyldecyl (m=6, n=8), 2-octyldecyl (m=8, n=8), 2-hexyldodecyl (m=6, n=10), 2-octyldodecyl (m=8, n=10), 2-decyltetradecyl (m=10, n=12) or 2-dodecylhexadecyl (m=12, n=14) radical. Particularly preferably, the sum m+n is equal to 18, 22 or 26. Very particularly preferably, the sum m+n is equal to 22 or 26.

In the formula R—O—(X)ₚ, the group R—O— is bonded to X by the anomeric carbon of the xylose residue to form an acetal group.

p, which represents the mean degree of polymerization of the xylose, is more particularly between 1 and 2.5 and very particularly between 1 and 2.0.

The compound of the formula R—O—(X)ₚ can be prepared by reacting xylose with an excess of a fatty alcohol of the formula ROH and then removing the unreacted fatty alcohol.

In the process as defined above, the reaction is carried out in the presence of strong acid catalysts.

In one variant of the process as defined above, the xylose is reacted with an alcohol of the formula R₁—OH, in which R₁ contains from 1 to 4 carbon atoms, and more particularly with butanol, to give the acetal of the formula R₁O—(X)ₚ, which is then subjected to a transacetalization with an excess of an alcohol of the formula ROH, with distillation of the alcohol of the formula R₁OH formed, followed by removal of the unreacted alcohol of the formula ROH.

In this process and its variant as described above, the unreacted alcohol of the formula ROH is removed by methods known to those skilled in the art, for example by distillation, thin film distillation, molecular distillation or solvent extraction, or via supercritical fluids.

According to a second feature, the invention relates to a water-in-oil emulsion comprising from 0.2 to 25% by weight, preferably from 0.2 to 10% by weight and particularly preferably from 0.5 to 5% by weight of one or more alkylpolyxylosides as defined above.

In one advantageous embodiment, each alkylpolyxyloside of formula (I) is mixed with its corresponding Guerbet alcohol (of the formula ROH, where R is as defined above) in an alkylpolyxyloside/alcohol weight ratio ranging from 1/99 to 99/1.

The water-in-oil emulsion generally comprises from 5 to 90% by weight and preferably from 10 to 70% by weight of one or more oils selected in particular from:

oils of vegetable origin such as sweet-almond oil, copra oil, castor oil, jojoba oil, olive oil, colza oil, groundnut oil, sunflower oil, wheat germ oil, maize oil, soybean oil, cottonseed oil, alfalfa oil, poppy oil, pumpkin oil, evening primrose oil, millet oil, barley oil, rye oil, safflower oil, candlenut oil, passion flower oil, hazelnut oil, palm oil, shea butter, apricot kernel oil, calophyllum oil, sysymbrium oil, avocado oil and calendula oil;

vegetable oils and their ethoxylated methyl esters;

oils of animal origin such as squalene and squalane;

mineral oils such as paraffin oil, liquid petrolatum and isoparaffins; and

synthetic oils, especially fatty acid esters such as butyl myristate, propyl myristate, cetyl myristate, isopropyl palmitate, butyl stearate, hexadecyl stearate, isopropyl stearate, octyl stearate, isocetyl stearate, dodecyl oleate, hexyl laurate and propylene glycol dicaprylate, esters derived from lanolic acid, such as isopropyl lanolate and isocetyl lanolate, fatty acid monoglycerides, diglycerides and triglycerides such as glycerol triheptanoate, alkyl benzoates, polyalphaolefins, polyolefins such as polyisobutene, synthetic isoalkanes such as isohexadecane and isododecane, perfluorinated oils and silicone oils. Among the latter, those which may be mentioned more particularly are dimethylpolysiloxanes, methylphenylpolysiloxanes, silicones modified by amines, silicones modified by fatty acids, silicones modified by alcohols, silicones modified by alcohols and fatty acids, silicones modified by polyether groups, epoxy-modified silicones, silicones modified by fluoro groups, cyclic silicones and silicones modified by alkyl groups.

This oil can also be selected from fatty acids, fatty alcohols, waxes of natural or synthetic origin and, more generally, any fats of vegetable, animal or synthetic origin.

The water-in-oil emulsion according to the present invention can also optionally contain up to 10% by weight of a coemulsifier and up to 10% by weight of a stabilizer.

Stabilizers which may be mentioned among those capable of being used within the framework of the present invention are hydrogenated castor oil; vegetable or animal waxes, for example beeswax and carnauba wax; stearic acid and its metal salts such as aluminum stearate; hydrophobic silicas; polyethylene glycol/alkyl glycol copolymers, for example a PEG-45/dodecyl glycol copolymer such as the product marketed under the name ELFACOS ST 9®; polymers such as the products marketed under the name KRATON®; mineral waxes such as ozokerite; clays such as hectorite or bentonite; and hydrophobic modified starches, for example the product marketed under the name DRY FLOW PC®.

Coemulsifiers which may be mentioned in particular among those capable of being used within the framework of the present invention are lipoamino acids and their salts; lipopeptides and their salts; non-ionic and anionic silicone-containing emulsifiers; sorbitan esters; polyglycerol esters; ethoxylated hydrogenated castor oil; glycerol stearate; polyol polyhydroxystearates, for example the product called HYPERMER® B241; cationic emulsifiers, for example amine oxides and quatemium 82; ethoxylated or non-ethoxylated sucrose esters and methylglucoside esters; ethoxylated fatty acids; ethoxylated fatty alcohols; and anionic emulsifiers such as decylphosphate or cetearylsulfate.

The water-in-oil emulsion also advantageously comprises one or more mineral salts, for example magnesium chloride, magnesium sulfate, sodium borate or sodium chloride, in an amount ranging from 0.1% to 5% by weight.

The water-in-oil emulsions according to the present invention can be prepared simply by dispersing the aqueous phase in the oily phase at a temperature of between 15° C. and 90° C., in the presence of the emulsifier(s) and optionally the stabilizer(s).

In a manner known per se, these emulsions can also comprise one or more compounds selected from humectants, for example glycerol; preservatives, for example the products known under the name SEPICIDE®; colorants; perfumes; cosmetic active ingredients; mineral or organic sunscreen agents; mineral fillers such as iron oxides, titanium oxides and talcum; synthetic fillers such as nylons and crosslinked or non-crosslinked polymethyl methacrylates; silicone elastomers; sericites; and plant extracts.

These compounds may be introduced into the aqueous phase or into the oily phase, depending on their affinity for these phases, either during the aforementioned dispersion phase or, as regards the temperature-sensitive compounds, subsequently during the cooling phase in the case where the dispersion is prepared under the action of heat.

The invention will be illustrated by the Examples which follow.

### EXAMPLE 1

### Preparation of a 2-octyldecylxyloside and a 2-hexyldodecylxyloside

A mixture of alcohols comprising predominantly 2-hexyldodecanol and 2-octyldecanol, marketed under the name Isofol® 18 by SASOL, is heated to 90° C. in a reactor, xylose is then added in a xylose/alcohol stoichiometric ratio of 1/6, with stirring, and the mixture is left to react for 4 hours in the presence of an acid catalyst. The product obtained after cooling, neutralization and filtration has a hydroxyl number of 241 and contains 15% by weight of a mixture of 2-octyldecylxyloside and 2-hexyldodecylxyloside and 85% by weight of a mixture of 2-octyldecanol and 2-hexyldodecanol.

### EXAMPLE 2

### Preparation of a 2-octyldecylxyloside and a 2-hexyldodecylxyloside

The product obtained in Example 1 is partially distilled to give a product containing 60% by weight of a mixture of 2-octyldecylxyloside and 2-hexyldodecylxyloside and 40% by weight of a mixture of 2-octyldecanol and 2-hexyldodecanol.

### EXAMPLE 3

### Preparation of a 2-octyldecylxyloside and a 2-hexyldodecylxyloside

The product obtained in Example 1 is totally distilled to give 100% of a mixture of 2-octyldecylxyloside and 2-hexyldodecylxyloside.

### EXAMPLE 4

### Preparation of a 2-decyltetradecylxyloside

61.8 kg of 2-decyltetradecanol, marketed by SASOL under the name Isofol® 24, are introduced into a reactor. 8.7 kg of xylose are gradually dispersed in the stirred medium, after which 65 g of sulfuric acid are added. The mixture is maintained at 115° C. for 6 hours under a partial vacuum and then neutralized with sodium hydroxide solution. The clear liquid obtained after filtration has a hydroxyl number of 183 and contains 15% by weight of 2-decyltetradecylxyloside and 85% by weight of 2-decyltetradecanol.

### EXAMPLE 5

### Preparation of a 2-dodecylhexadecylxyloside

Under the same stoichiometric and reaction conditions as Example 4, 2-dodecylhexadecanol, marketed by SASOL under the name Isofol® 28, is reacted with xylose to give a liquid having a hydroxyl number of 141 and containing 15% by weight of 2-dodecylhexadecylxyloside and 85% by weight of 2-dodecylhexadecanol.

### COMPARATIVE EXAMPLE 1

### Preparation of an Isostearylglucoside

Isostearyl alcohol (the product marketed by UNIQEMA under the name PRISORINE® 3515 or by COGNIS under the name SPEZIOL® C18 ISO) is introduced into a reactor.

Glucose is also introduced into the reactor so that the molar ratio of isostearyl alcohol to glucose is 6/1.

The glucose is then reacted with the fatty alcohol for 6 hours at a temperature of about 100° C. under a partial vacuum, in the presence of an acid catalyst.

When the reaction is complete, the catalyst is neutralized with a base.

The product obtained contains 15% by weight of isostearylglucoside and 85% by weight of isostearyl alcohol.

### COMPARATIVE EXAMPLE 2

### Preparation of an Isostearylxyloside

The procedure of Comparative Example 1 is followed except that the glucose is replaced with xylose.

The product obtained contains 15% by weight of isostearylxyloside and 85% by weight of isostearyl alcohol.

### COMPARATIVE EXAMPLE 3

### Preparation of a 2-octyldecylglucoside and a 2-hexyldodecylglucoside

The procedure of Example 1 is followed except that the xylose is replaced with glucose.

The product obtained contains 15% by weight of a mixture of 2-octyldecylglucoside and 2-hexyldodecylglucoside and 85% by weight of a mixture of 2-octyldecanol and 2-hexyldodecanol.

### EXAMPLE 6

### Demonstration of the Emulsifying Properties on Paraffin Oil

The emulsions are prepared in the following manner:

The aqueous phase is heated to a temperature of 70 to 85° C. In parallel, the oily phase, containing the emulsifying system of the invention and the oils, is heated to an identical temperature of 70 to 85° C.

The 2 phases are then mixed and emulsified using a rotor-stator emulsifying mixer (for example a SILVERSON laboratory mixer for 4 min at 4000 rpm). After emulsification, the emulsion is cooled with moderate stirring.

All the emulsions obtained are of the water-in-oil type.

The results are shown in Table 1.

These results illustrate:

the absence of emulsifying power in the case of the alkylpolyglucoside obtained from a Guerbet alcohol;

the better stability of the emulsions according to the invention obtained with alkylpolyxylosides derived from a Guerbet alcohol compared with the alkylpolyglucosides and alkylpolyxylosides derived from isostearyl alcohol.

### EXAMPLE 7

### Demonstration of the Emulsifying Properties on a C8—C10 Triglyceride

The emulsions are prepared by the procedure of Example 6. All the emulsions obtained are of the water-in-oil type.

The results are shown in Table 2.

Here again the results illustrate:

the absence of performance in the case of the alkylpolyglucoside obtained from a Guerbet alcohol;

the superior performance of the alkylpolyxylosides according to the invention compared with the emulsifiers of Comparative Examples 1 and 2.

### EXAMPLE 8

### Demonstration of the Emulsifying Properties on Squalane

The emulsions are prepared by the procedure of Example 6. All the emulsions obtained are of the water-in-oil type.

The results are shown in Table 3.

Here again the results illustrate:

the absence of performance in the case of the alkylpolyglucoside obtained from a Guerbet alcohol;

the superior performance of the alkylpolyxylosides according to the invention compared with the emulsifiers of Comparative Examples 1 and 2.

### EXAMPLE 9

### Demonstration of the Emulsifying Properties on Cetearyl Octanoate

The emulsions are prepared by the procedure of Example 6. All the emulsions obtained are of the water-in-oil type.

The results are shown in Table 4.

Here again the results illustrate:

the absence of performance in the case of the alkylpolyglucoside obtained from a Guerbet alcohol;

the superior performance of the alkylpolyxylosides according to the invention compared with the emulsifiers of Comparative Examples 1 and 2.

### EXAMPLE 10

### Demonstration of the Emulsifying Properties in Association With Other Emulsifiers

The emulsions are prepared in the following manner:

The 2 phases are mixed without prior heating and emulsified using a rotor-stator emulsifying mixer (of the SILVERSON laboratory mixer type for 4 min at 4000 rpm).

All the emulsions obtained are of the water-in-oil type.

The results are shown in Table 5.

Even in association with other lipophilic emulsifiers, the performance of the alkylpolyxylosides according to the invention is superior to that of the emulsifiers of Comparative Examples 1 and 2.

TABLE 1

| | | | | | | Emulsion | Emulsion | |
|---|---|---|---|---|---|---|---|---|
| | | | | | | with | with | |
| | Emulsion | Emulsion | Emulsion | Emulsion | Emulsion | Comp. | Comp. | Emulsion with |
| | with Ex. 1 | with Ex. 2 | with Ex. 3 | with Ex. 4 | with Ex. 5 | Ex. 1 | Ex. 2 | Comp. Ex. 3 |
| 2-Octyldecylxyloside and | 1.2 | 1.2 | 1.2 | | | | | |
| 2-hexyldodecylxyloside | | | | | | | | |
| 2-Decyltetradecylxyloside | | | | 1.2 | | | | |
| 2-Dodecylhexadecylxyloside | | | | | 1.2 | | | |
| Isostearylglucoside | | | | | | 1.2 | | |
| Isostearylxyloside | | | | | | | 1.2 | |
| 2-Octyldecylglucoside and | | | | | | | | 1.2 |
| 2-hexyldodecylglucoside | | | | | | | | |
| 2-Octyldecanol and | 6.8 | 1.8 | | | | | | 6.8 |
| 2-hexyldodecanol | | | | | | | | |
| Decyltetradecanol | | | | 6.8 | | | | |
| Dodecylhexadecanol | | | | | 6.8 | | | |
| Isostearyl alcohol | | | | | | 6.8 | 6.8 | |
| PEG 45/dodecyl glycol copolymer | 2 | 2 | 2 | 2 | 2 | 2 | 2 | 2 |
| Paraffin oil | 40 | 45 | 46.8 | 40 | 40 | 40 | 40 | 40 |
| Glycerol | 5 | 5 | 5 | 5 | 5 | 5 | 5 | 5 |
| Water | 44.3 | 44.3 | 44.3 | 44.3 | 44.3 | 44.3 | 44.3 | 44.3 |
| MgSO₄.7H₂O | 0.7 | 0.7 | 0.7 | 0.7 | 0.7 | 0.7 | 0.7 | 0.7 |
| Stability | Exudation | Exudation | Exudation | >6 months | >6 months | Exudation | Exudation | No |
| | of oil after | of oil after | of oil after | | | of oil after | of oil after | emulsification |
| | 6 months | 6 months | 6 months | | | 1 month | 3 months | |

TABLE 2

| | Emulsion with | Emulsion with | Emulsion with Comp. | Emulsion with | Emulsion with Comp. |
|---|---|---|---|---|---|
| | Ex. 1 | Ex. 4 | Ex. 1 | Comp. Ex. 2 | Ex. 3 |
| 2-Octyldecylxyloside and | 1.2 | | | | |
| 2-hexyldodecylxyloside | | | | | |
| 2-Decyltetradecylxyloside | | 1.2 | | | |
| Isostearylglucoside | | | 1.2 | | |
| Isostearylxyloside | | | | 1.2 | |
| 2-Octyldecylglucoside and | | | | | 1.2 |
| 2-hexyldodecylglucoside | | | | | |
| 2-Octyldecanol and | 6.8 | | | | 6.8 |
| 2-hexyldodecanol | | | | | |
| 2-Decyltetradecanol | | 6.8 | | | |
| Isostearyl alcohol | | | 6.8 | 6.8 | |
| PEG 45/dodecyl glycol copolymer | 2 | 2 | 2 | 2 | 2 |
| C8-C10 triglyceride | 40 | 40 | 40 | 40 | 40 |
| Glycerol | 5 | 5 | 5 | 5 | 5 |
| Water | 44.3 | 44.3 | 44.3 | 44.3 | 44.3 |
| MgSO₄.7H₂O | 0.7 | 0.7 | 0.7 | 0.7 | 0.7 |
| Stability | >6 months | >6 months | No emulsification | Exudation of oil | No emulsification |
| | | | | after 7 days | |

TABLE 3

| | Emulsion with | Emulsion with | Emulsion with | Emulsion with | Emulsion with Comp. |
|---|---|---|---|---|---|
| | Ex. 1 | Ex. 4 | Comp. Ex. 1 | Comp. Ex. 2 | Ex. 3 |
| 2-Octyldecylxyloside and | 1.2 | | | | |
| 2-hexyldodecylxyloside | | | | | |
| 2-Decyltetradecylxyloside | | 1.2 | | | |
| Isostearylglucoside | | | 1.2 | | |
| Isostearylxyloside | | | | 1.2 | |
| 2-Octyldecylglucoside and | | | | | 1.2 |
| 2-hexyldodecylglucoside | | | | | |
| 2-Octyldecanol and 2-hexyldodecanol | 6.8 | | | | 6.8 |
| Decyltetradecanol | | 6.8 | | | |
| Isostearyl alcohol | | | 6.8 | 6.8 | |
| PEG 45/dodecyl glycol copolymer | 2 | 2 | 2 | 2 | 2 |
| Squalane | 40 | 40 | 40 | 40 | 40 |
| Glycerol | 5 | 5 | 5 | 5 | 5 |
| Water | 44.3 | 44.3 | 44.3 | 44.3 | 44.3 |
| MgSO₄.7H₂O | 0.7 | 0.7 | 0.7 | 0.7 | 0.7 |
| Stability | Exudation of oil | >6 months | Exudation of oil | Exudation of oil | No emulsification |
| | after 6 months | | after 1 month | after 1 month | |

TABLE 4

| | Emulsion with | Emulsion | Emulsion with Comp. | Emulsion with | Emulsion with Comp. |
|---|---|---|---|---|---|
| | Ex. 1 | with Ex. 4 | Ex. 1 | Comp. Ex. 2 | Ex. 3 |
| 2-Octyldecylxyloside and | 1.2 | | | | |
| 2-hexyldodecylxyloside | | | | | |
| 2-Decyltetradecylxyloside | | 1.2 | | | |
| Isostearylglucoside | | | 1.2 | | |
| Isostearylxyloside | | | | 1.2 | |
| 2-Octyldecylglucoside and | | | | | 1.2 |
| 2-hexyldodecylglucoside | | | | | |
| 2-Octyldecanol and | 6.8 | | | | 6.8 |
| 2-hexyldodecanol | | | | | |
| Decyltetradecanol | | 6.8 | | | |
| Isostearyl alcohol | | | 6.8 | 6.8 | |
| PEG 45/dodecyl glycol copolymer | 2 | 2 | 2 | 2 | 2 |
| Cetearyl octanoate | 40 | 40 | 40 | 40 | 40 |
| Glycerol | 5 | 5 | 5 | 5 | 5 |
| Water | 44.3 | 44.3 | 44.3 | 44.3 | 44.3 |
| MgSO₄.7H₂O | 0.7 | 0.7 | 0.7 | 0.7 | 0.7 |
| Stability | Exudation of oil | >6 months | No emulsification | Exudation of oil | No emulsification |
| | after 6 months | | | after 3 months | |

TABLE 5

| | Emulsion | Emulsion with | Emulsion with | Emulsion | Emulsion with | Emulsion with |
|---|---|---|---|---|---|---|
| | with Ex. 4 | Comp. Ex. 1 | Comp. Ex. 2 | with Ex. 4 | Comp. Ex. 1 | Comp. Ex. 2 |
| 2-Decyltetradecylxyloside | 0.9 | | | 0.9 | | |
| Isostearylglucoside | | 0.9 | | | 0.9 | |
| Isostearylxyloside | | | 0.9 | | | 0.9 |
| 2-Decyltetradecanol | 5.1 | | | 5.1 | | |
| Isostearyl alcohol | | 5.1 | 5.1 | | 5.1 | 5.1 |
| C8-C10 triglyceride | 40 | 40 | 40 | 40 | 40 | 40 |
| PEG 150 polyhydroxystearate | 4 | 4 | 4 | | | |
| Dimethicone copolyol | | | | 4 | 4 | 4 |
| Glycerol | 5 | 5 | 5 | 5 | 5 | 5 |
| Water | 44.3 | 44.3 | 44.3 | 44.3 | 44.3 | 44.3 |
| MgSO₄.7H₂O | 0.7 | 0.7 | 0.7 | 0.7 | 0.7 | 0.7 |
| Stability | >6 months | Exudation of oil | Exudation of | >6 months | No emulsification | Exudation of |
| | | after 7 days | oil after 1 | | | oil after 7 days |
| | | | month | | | |

1. Use of an alkylpolyxyloside or a mixture of alkylpolyxylosides of the formula
R—O—(X)ₚ (I)
in which:
p is a decimal number between 1 and 5,
X is the xylose residue, and
R is a branched alkyl radical of the formula
CH(CₙH₂ₙ₊₁)(CₘH₂ₘ₊₁)—CH₂—
in which m is an integer between 6 and 12, n is an integer between 8 and 14 and the sum n+m ranges from 14 to 26,
as an emulsifier for the preparation of water-in-oil emulsions.

2. Use according to claim 1 in which the sum m+n in formula (I) is equal to 14, 16, 18, 22 or 26, preferably equal to 18, 22 or 26 and particularly preferably equal to 22 or 26.

3. Use according to claim 1 or 2 in which R in formula (I) is a 2-hexyldecyl, 2-octyldecyl, 2-hexyldodecyl, 2-octyldodecyl, 2-decyltetradecyl or 2-dodecylhexadecyl radical.

4. Water-in-oil emulsion comprising from 0.2 to 25% by weight, preferably from 0.2 to 10% by weight and particularly preferably from 0.5 to 5% by weight of an alkylpolyxyloside or a mixture of alkylpolyxylosides of the formula
R—O—(X)ₚ (I)
in which:
p is a decimal number between 1 and 5,
X is the xylose residue, and
R is a branched alkyl radical of the formula
CH(CₙH₂ₙ₊₁)(CₘH₂ₘ₊₁)—CH₂—
in which m is an integer between 6 and 12, n is an integer between 8 and 14 and the sum n+m ranges from 14 to 26.

5. Emulsion according to claim 4 in which each alkylpolyxyloside is mixed with its corresponding alcohol of the formula ROH in an alkylpolyxyloside/alcohol weight ratio ranging from 1/99 to 99/1.

6. Emulsion according to claim 4 or 5 comprising from 5 to 90% by weight and preferably from 10 to 70% by weight of one or more oils.

7. Emulsion according to one of claims 4 to 6 in which the sum m+n in formula (I) is equal to 14, 16, 18, 22 or 26, preferably equal to 18, 22 or 26 and particularly preferably equal to 22 or 26.

8. Emulsion according to one of claims 4 to 7 in which R in formula (I) is a 2-hexyldecyl, 2-octyldecyl, 2-hexyldodecyl, 2-octyldodecyl, 2-decyltetradecyl or 2-dodecylhexadecyl radical.
